(19) United States
(12) Patent Application Publication
(10) Pub. No.: US 2026/0232560 A1
(43) Pub. Date: Aug. 13, 2026

(54) SKIN HEALTH BENEFITTING COMPOSITIONS

(71) Applicants: SHANKARANARAYANAN JEYAKODI, HYDERABAD (IN); ARUNKANTH KRISHNAKUMARAN NAIR, HYDERABAD (IN)

(72) Inventors: SHANKARANARAYANAN JEYAKODI, HYDERABAD (IN); ARUNKANTH KRISHNAKUMARAN NAIR, HYDERABAD (IN)

(21) Appl. No.: 19/155,693

(22) PCT Filed: Feb. 15, 2024
(86) PCT No.: PCT/IN2024/050151

§ 371 Date: Aug. 12, 2025

(30) Foreign Application Priority Data
Feb. 15, 2023 (IN) 202241065480

(51) Int. Cl. A61K 8/60; A61K 8/02; A61K 8/11; A61K 8/36; A61K 8/49; A61K 8/73; A61Q 19/08

(52) U.S. Cl. CPC A61K 8/60; A61K 8/022; A61K 8/11; A61K 8/361; A61K 8/498; A61K 8/73; A61K 8/731; A61K 8/732; A61Q 19/08; A61K 2800/92

(57) ABSTRACT

The present embodiment relates to a composition comprising ellagic acid, punicalagin and punicic acid in an optimized ratio to provide therapeutic benefits to a healthy lifestyle. In addition, the present embodiment also relates to a process for combining powdered extract of ellagic acid and punicalagin with an oil containing punicic acid and using a proprietary technology of spray drying to achieve encapsulation using a natural plant-based gum to arrive at a substantially aqueous soluble product thus enhancing bioavailability.

### TECHNICAL FIELD

The present disclosure generally relates to enhancing physicochemical properties of pomegranate, more particularly relates to enhancing bio-accessibility of a soluble punicic acid compound. Further, the present invention relates to a process for preparing the soluble composition.

### BACKGROUND OF THE INVENTION

Punica granatum: Punica granatum Linn. (Pomegranate) is a fruit plant species of Punicaceae family locally known as Anar. It is a plant of great antiquity cultivated in the Middle East more than 5,000 years ago. The plant is found all over India and Bangladesh. In Ayurvedic medicine, the plant is described under its Sanskrit name “dalima” (fruit) as a “blood purifier” and used to cure parasitic infections, aphthae (mouth ulcers), diarrhoea, and ulcers. The fruit rind of P. granatum is rich in beta-carotene, potassium, phosphorous, calcium and various chemical constituents like ellagic acid, punicalagins, ellagitannins, punicic acid, flavonoids, anthocyanidins, anthocyanins and flavones. The potential wide-ranging therapeutic properties of Pomegranate rind or peel include treatment and prevention for cancer, cardiovascular disease, diabetes, dental conditions, and erectile dysfunction, protection from ultraviolet radiation; and as anti-diarrheal and antimicrobial. Other potential applications include infant brain ischemia, Alzheimer's disease, male infertility, arthritis, dermal wounds and obesity.

### Phytochemistry:

Pomegranate pericarp has a high concentration of phytochemicals, mainly polyphenolic flavonoids, and ellagitannins, containing ellagic acid and punicalagin. Pomegranate contains phytochemical compounds in its diverse parts, which have several pharmacological functions.

Pomegranate juice contains Anthocyanin creates an attractive purple-red colour in pomegranate juice. The amounts of fatty acids, sterols, organic acids, triterpenoids, and minerals content have been also reported in pomegranate juice. In general, pomegranate has two main composition groups:

Hydrolysable tannins are the principal group of pomegranate polyphenols, which consist of gallotannins, ellagitannins, galagyl esters, hydroxycinnamic acids, and hydroxybenzoic acids.

The prime ingredient of ellagitannins is punicalagin, which is mostly recognized in the pericarp, peel, flowers and seeds. In addition, punicalagin and its isomers, pomegranate contains punicalin A, punicalin B, and pedunculagin isomers. Likewise, Gallic acid, ellagic acid, caffeic acid, chlorogenic acid, p-coumaric acid, aglycone, and ferulic acid are present in pomegranate.

### Anthocyanosides;

Anthocyanosides are the next significant ingredient group that exists in the flower, fruit, and are accountable for the red colour of arils. Pomegranate is rich in anthocyanin, which has significant antioxidant capacity. Anthocyanins mainly act as primary antioxidants.

### Ellagic Acid (EA):

Ellagic acid, a polyphenol compound present in berries and pomegranate, has received attention as an agent that may have potential bioactivities preventing chronic diseases. (Ji-Young Bae et al., Exp Dermatol. 2010 August; 19(8):e182-90).

Apart from the antioxidant activity, ellagic acid has skin whitening effect and reduces UV-induced skin pigmentation, which is due to inhibition of tyrosinase activity by binding to copper at its active site, Ellagic acid can also be used in antidandruff shampoo preparations. Formulation containing ellagic acid can be used as antipollution cosmetic agent, which protects the skin from detrimental effect of pollution. Ellagic acid-containing formulation has been reported for treating greying of the hair. (Kuttuvan Valappil Sajna et al. Industrial Biorefineries & White Biotechnology, 2015).

It has been reported that ellagic acid has a high affinity for copper at the active site of tyrosinase and inhibits its activity. Shimogaki et al. have reported that when ellagic acid was topically applied, it suppressed UV-induced skin pigmentation of brownish guinea pigs. (Mineka Yoshimura et al. Biosci, Biotechnol. Biochem., 69 (12), 2368-2373, 2005)

Ellagic acid prevented proteolytic degradation of existing dermal elastic fibres and efficiently enhanced elastogenesis in aged skin. Pomegranate juice has the highest concentration of ellagitannins among commonly consumed juices and contains the unique polyphenol ellagitannin, punicalagin. Ellagitannins are not absorbed intact into the blood stream but are hydrolysed to ellagic acid. Pomegranate Fruit Extract exhibited anti-oxidant properties, which was thought to be because of the action of ellagic acid.

Ji-Young Bae et al demonstrated ellagic acid diminished the release of pro-inflammatory cytokines in hairless mouse skin chronically exposed to UV-B. The inhibition of cytokine release by ellagic acid was most likely responsible for reduction of collagenolytic MMP production and subsequent mitigation of collagen fibre degradation.

### Pomegranate Seed Oil (PSOE):

PSO contains punicic acid, linoleic acid, %, oleic acid, gadoleic acid, erucic acid, linolenic acid, and palmitoleic acid.

Although pomegranate seeds, which represent about 3% of the fruit weight, have a low polyphenol content and in vitro antioxidant capacity, they contain other components that may contribute to pomegranate's health benefits. They are a rich source of lipids, and their oil, which constitutes 12-20% of total seed weight, contains a unique fatty acid profile characterized by high concentration of fatty acids such as linoleic acid (LA) and linolenic acid (LN), as well as other lipids including punicic, oleic, stearic, α-eleostearic, β-eleostearic, catalpic, gadoleic, arachidic, behenic, and palmitic acids LN. Interestingly, punicic acid, which is a conjugated isomer unique to pomegranate oil, constitutes 70-76% of the seed oil.

### Pomegranate Peels:

Pomegranate skin has various forms of tannins, which are water-soluble and hydrolysable phenolics. According to the structure, tannins have four main categories: gallotannins, ellagitannins, complex tannins, and dense tannins. Punicalagin is an ellagitannin which is the major component in the peel of pomegranate, whose content is much higher than other compounds.

In traditional medicine, pomegranate peel, flowers, branches, and roots are used. All P. granatum L. components have strong astringent effects due to the presence of abundant tannins. Several pomegranate flower infusions and decoctions of the flowers have been utilized to remedy diarrhoea and vaginal infectious discharge. In addition, the extract of pomegranate peel has been used to treat acute pancreatitis.

Fresh pomegranate juice is used for remedy of gall bladder problems and its decoction is useful for any types of diarrhoeas, either simple or bloody diarrhoea and stomach discomfort. Alkaloid substances in pomegranate root bark and ethanolic extract are used to eliminate intestinal parasites.

### Antioxidant Activity:

Polyphenol hydroxyl groups in pomegranate are responsible for antioxidant activity by scavenger activity free radicals. Likewise, the catechol hydroxyl groups of complex and condensed tannins have the potency to chelate iron and transition metals.

Pomegranate is a rich of anthocyanin, which has a potent antioxidant capacity. Gill et al. found that phenolic ingredients are responsible for 28% of the total antioxidant function of pomegranate, which indicates the important role of anthocyanin in its antioxidant.

There are several articles about the antioxidant activity of pomegranate, mostly based on the DPPH assay. In general, the parts of pomegranate have significant anti-radical and anti-microbial effects, which can make this plant introduced safe as an herbal source of preservatives and antioxidants. Therefore, P. granatum might be used as an auxiliary substance as mentioned above in pharmaceutical and food industries.

### Skin Whitening Activity

Topical microemulsion containing P. granatum extract controls erythema and redness of skin as well as skin melanin in people. Since P. granatum is rich in polyphenolic compounds, it shows its function with strong activity in inhibiting free radicals. It is believed that ellagic acid acts as a skin brightener by chelating copper in tyrosinase. This research shows that the microemulsion of P. granatum extract could be utilized in a topical formulation for skin spots and erythema.

### Anti-Acne Activity

Acne vulgaris is a prevalent dermatology disease associated by excessive sebum production, hyperkeratosis, and the presence of Cutibacterium acnes, Staphylococcus aureus, and inflammation. Lee et al. indicated that pomegranate extract remarkably decreased swelling by C. acnes of the Wistar rats' ears.

Their results represented that pomegranate extract inhibited bacterial growth and lipase function. Four hydrolysable tannins are punicalagin, punicalin, strytinin A, and also granatin B. Punicalagin and punicalin compounds had antibacterial properties and testosterone-induced HaCaT proliferative effects more than others. Punicalagin, strictinin, and granatin B showed lipase inhibitory effects. Granatin B compound causes downregulation of cyclooxygenase-2 expression and prostaglandin E2 production in RAW 246.7 cells treated with P. acnes.

As a result, pomegranate hydrolysable tannins show strong anti-acne, anti-lipase, anti-keratinocyte proliferation, and anti-inflammatory activities. Therefore, pomegranate extract (PG-E) has major potential for topical use in anti-acne agents, and punicalagin appears to be the most effective pomegranate compound and quality control indicator.

### Anti-Skin Aging Activity

The Ultraviolet (UV) radiation causes various skin problems, including sunburn, inflammation, hyperplasia, immune system suppression, skin aging, and skin cancer. Using the GC-MS method, Park et al, showed that pomegranate polyphenols help to prevent and protect the adverse skin effects of ultraviolet rays. To consider the protective pomegranate activity of skin aging caused by UVB, they determined the level of procollagen type I and MMP-1. Finally, the dominant compounds were catechin, quercetin, kaempferol, and equol, Sunlight induces changes in the expression of procollagen type I and MMP-1 in fibroblasts. Studies showed that pomegranate catechins, which are one of its main polyphenol compounds, can play a main role in protecting against skin damage caused by UVB.

Many in vivo and in vitro tests have been indicated that polyphenols have antioxidant, anti-inflammation, and anti-cancer activity. It seems that pomegranate fruit extract standardized to punicalagins had effective in keep fibroblasts apoptosis after UV radiation, probably by reducing the activation of pro-inflammation transcription factor NF-kappa B a downregulation of proapoptotic caspase-3, and an increased G0/G1 phase, linked to DNA repair. The results of this survey illustrated the protecting properties of pomegranate extract versus UVA and UVB-induced apoptosis and the possible application of extract polyphenols for topical formulas.

The pomegranate fruit extract has a potent antioxidant and anti-inflammation hallmark. The current research has indicated that pomegranate fruit extract treatment of human epidermal keratinocytes prevents UVB-mediated activation of MAPK and NF-kappa B pathways. Signal transducers and activators of transcription 3 (STAT3), Protein Kinase B/AKT, and Map Kinases (MAPKs) regulate cell proliferation, apoptosis, and other biologic pathways, Pomegranate protects skin against UVA-mediated activation of STAT3, AKT, and extracellular signal-regulated kinase (ERK1/2). Our data indicated that PG is an effective agent for ameliorating UVA-induced damage and it is worth further evaluation as a photo preventive agent.

### Anti-Stretch Marks

Stretch Marks called as dermal lesions with multifactorial mechanisms had not been precisely explicated so far. However, striae scarcely cause serious medical issues, they present a cosmetic concern for sufferers, Bogdan C. et al.'s study on the clinical effectiveness of a PSO extract and C. lechleri cream formulation indicated that it could be useful in preventing or improving striae.

### Hair Growth Promoting Activity, Anti-Dandruff, and Cure of Head Lice:

It seems that use of Punica granatum in skin and hair diseases as well as cosmetic industry may be beneficial. According to a new research, pomegranate extract may be useful for prevention of hair loss, as anti-dandruff, and cure of head lice. New research is need for potential use for other application of this precious local plant.

The main substance in pomegranate oil is punicic acid (PA), Punicic acid (also known also as trichosanic acid), is a conjugated linolenic acid isomer containing cis-9, trans-11, cis-13 double bonds in the C 18 carbon chain. In this context, U.S. patent application Ser. No. 11/039,419 discusses the use of punicic acid to enhance immune response and prevent metabolic disorders. Punicic acid (PA) has 4 mechanisms of action. First, it is a powerful antioxidant approximately 10 times greater than that of grape seed extract. Second, PA is a conjugated linolenic acid (CLA). There is considerable interest in CLAs because they exhibit anti-inflammatory, anti-plaque in blood vessels, and antitumor properties. Most CLAs come from animal sources. PA is the only medicinal oil that is a useable CLA that comes from a plant source. CLAs bind to receptors on the nucleus of cells that regulate the production of glucose transport channels. Therefore, CLA is important in the control of glucose transport at the cell surface. Third, PA has three double bonds in the 9 cis, 11 trans, and 13 cis, position. These double bonds bend the fatty acid chain in a way that resembles arachidonic acid. Arachidonic acid is powerful pro-inflammatory fatty acid that is the precursor of inflammatory prostaglandins that produce disease. PA inhibits the production of arachidonic acid and down regulates the production of prostaglandins and leukotrienes that cause disease without adverse effects like those caused by non-steroidal anti-inflammatory drugs (aspirin e.g.). Fourth, PA has a profound effect on the electromagnetic field. Field energy science is a relatively new area of medicine and technology. Pomegranate oil is being investigated as an anti-tumour drug, especially prostate cancer in males and breast cancer in females worldwide. It is also being assessed as an anti-inflammatory drug in several immune complex disorders such as multiple sclerosis and systemic lupus erythematosus, and in in cardiovascular disease in the prevention of arterial plaque that results in heart attacks.

Pomegranate seed oil also contains in addition to punicic acid, palmitic acid, stearic acid, oleic acid, and linoleic acid. The human body can produce all but two of the fatty acids it needs. The two fatty acids that cannot be produced by the human body are linoleic acid and alpha linoleic acid, which are widely distributed in plant and fish oils. Since they cannot be made in the body from other substrates and must instead be supplied in food, they are called essential fatty acids. Essential fatty acids are polyunsaturated fatty acids, and are the parent compounds of the Omega-3, 5 and 6 fatty acid series, respectively. As noted above, the seed oil from pomegranates is an unsaturated fatty acid (punicic acid) and constitutes between 60-86% of the oil of the pomegranate fruit. Punicic acid is known to have an extremely strong ability to resist the oxidizing, inflammation, and destruction functions of the free radical of oxygen. As such, punicic acid may have wide application prospects in medicines and health protection, food, and the cosmetics industry.

Pomegranate seed oil is absorbed into the human skin, and from there into body cells. Therefore, it can be used to treat certain skin disorders as either a standalone product or when combined with the other ingredients. Given these unique properties, the pomegranate seed oil is an element of nanotechnology.

Most of the pomegranate extracts available in the market are standardised to either ellagic acid or punicalagin or polyphenols as bioactive. However, punicic acid is another potential active derived from pomegranate, which is basically in oil form and is not widely explored for its potential benefits. Pomegranate seed oil possesses rich polyunsaturated fatty acid predominantly comprising of punicic acid. To explore the complete potential of pomegranate as a wonder fruit, combining all three extracts at an optimal ratio is needed which shall be beneficial to the end customer yielding synergistic benefits.

Apparently, it is also evident from literatures that ellagic acid (EA) is highly insoluble in aqueous media, with limited bioavailability and absorption. Thus, there is a need for a processing technique wherein ellagic acid along with punicic acid and punicalagins should be formulated with improvised physicochemical properties eventually leading to better bioavailability, absorption, and therapeutic benefits. Such compositions and processes to manufacture such compositions are described herein.

### SUMMARY OF THE INVENTION

The present invention relates to a composition comprising different active constituents derived from pomegranate. The main active constituents are ellagic acid, punicalagins and punicic acid. Ellagic acid belongs to the class of BCS Class IV compound with poor solubility and permeability. It can be obtained from pomegranate fruit extracts in a powdered form and standardized extracts may contain up to 90% w/w of EA are reported in literature. Punicalagin also has poor aqueous solubility and is available in a powdered form and standardized extracts comprising around 90% w/w of punicalagin. Punicic acid is extracted from pomegranate seeds in oil form, Pomegranate oil may contain around 70-80% w/w of punicic acid. It is important that for holistic health benefits ideally all three bio actives play significant role. Considering the fact that two bio actives are extracted in a powder form whereas the third one is in an oily form it becomes challenging to combine these three active constituents in to a powder. In addition, an aqueous soluble composition is preferable because of its ease of administration and ease of formulating to desired dosage formats with better invitro in vivo characteristics.

The present invention also optimizes the ratio of ellagic acid, punicalagins and punicic acid to achieve a substantially stable and soluble product providing desired therapeutic benefits. The optimized ratios to achieve the desired therapeutic benefit may vary from 1:0.5:0.1 to 1:1.5:0.7 (ellagic acid:punicalagin:punicic acid). A preferred optimized ratio was combined extract to punicic acid 1:0.6:0.3 to 1:1.33:0.6.

Alternatively, a combined extract comprising ellagic acid and punicalagin may also be used wherein the ratio of the combined extract (ellagic acid+punicalagin) to punicic acid may vary from 1:0.1 to 1:0.5. A preferred optimized ratio was combined extract to punicic acid 1:0.15 to 1:0.3.

In terms of the concentration of actives in a composition, they may vary from 5-50% w/w of ellagic acid, 2-60% w/w punicalagins and 3-75% w/w of punicic acid.

The present invention also relates to a process for combining the powdered extracts of ellagic acid and punicalagin with the pomegranate seed oil extract containing punicic acid. Combining the ingredients wherein two are powdered extracts and one is an oily phase to achieve a triple bioactive free flowing powder is an onerous task. A proprietary encapsulation technology using a natural plant carrier like guar gum Arabica and starches was used to encapsulate the oil based active together with the powdered actives resulting in a free-flowing powder. This process thus converts and combines the two poorly aqueous soluble powdered extracts with the lipophilic oil extract to achieve a substantially water-soluble and stable powdered composition that can be easily incorporated into solid oral dosage forms like tablets, capsules, powders, gummies, oral shots, chews, ready to drink powder and beverages and incorporated into aqueous based creams.

The clinical trial conducted on the optimized composition improves overall skin appearance, skin glow, wrinkles and helps regulating hyper pigmentation. Further, present study underlines the positive effect of this unique ratio of bioactive composition in improving facial pores, spots, evenness, and skin elasticity levels at a dose of 300 mg.

### BRIEF DESCRIPTION OF DRAWINGS

FIG. 1 Graphs depicting changes in score for primary end-points during Visit 1 and Visit 4 in clinical trial study; and

FIG. 2 Graphs depicting changes in score for primary end-points during Visit 1 and Visit 4 in clinical trial study

### DETAILED DESCRIPTION OF THE INVENTION

The present embodiment describes a technique wherein actives in powder form as well as oil form are combined and processed into a uniform free flowing powder or granules or beadlets and standardized to form a composition containing ellagic acid, punicalagin and punicic acid at an optimal ratio.

In an example embodiment, a natural water-soluble plant-based carrier may be used as an encapsulating aid in the process which subsequently acts as a substrate to ensure the solubility and bioavailability of bio actives across the physiological pH range. In another embodiment, any other encapsulation aid such as modified starch can be used.

According to the literature it is assumed that systemic effects of EA might be hampered by its limited bioavailability, which could depend on i) its low solubility in aqueous media, ii) the in vivo hydrolysis of punicalagin to release EA, iii) the binding of EA and/or punicalagin to proteins which could decrease their accessibility, iv) the transport and absorption in the intestines. (Journal of Functional Foods; Volume 19, Part A, December 2015, Pages 225-235) It is known that Ellagic acid (EA) is highly insoluble in aqueous media, especially at acid pH, where most EA is not ionized (Bala et al, 2006, Hasegawa et al, 2003). This low solubility is partly due to its high degree of crystallinity, which is directly related to its planar and symmetrical structure and the hydrogen-bonding network formed in the crystal (Li, Harich, Wegiel, Taylor, & Edgara, 2013). Indeed, previous animal studies identified the low solubilityof EA in aqueous media as a major drawback in EA absorption (Daniel, Ratnayake, Kinstle, & Stoner, 1991) as well as its ability to bind21qa the intestinal epithelium (Whitley, Sweet, & Walle, 2006).

Therefore, it seems that ellagitannins are hydrolyzed in the stomach, where some ellagic acid is absorbed into circulation. The rest of ellagic acid is metabolized to urolithin derivatives by colonic microflora, and the less polar urolithin derivatives are then absorbed into circulation and further metabolized to glucuronides.

The proprietary ADOP processing technology developed to enhance the solubility of Ellagic acid and other bio actives in the gastric region and thereby enhancing the bioavailability. This technology will be improving the therapeutic benefits of pomegranate actives.

The unique attributes, biodegradability, biocompatibility, perfect accessibility, and low production costs led to the use of natural gums or starches as a carrier for many actives. Among them, we can use gums obtained from microorganisms (xanthan gum and gellan gum), plant tissues (Arabic gum and gum tragacanth), seeds (konjac gum and guar gum), seaweeds (alginates, agar gum, and carrageenan). Gums have essential applications in the medical and pharmaceutical, food, biotechnology, and critical agricultural industries. Encapsulation is one of the new methods to increase the stability of bioactive compounds during processing and storage.

### Some Natural Gums Used in Encapsulation:

1. Arabic Gum or Acacia: Arabic gum is the oldest and best-known natural gum secreted and obtained from the stems and branches of Acacia senegal or Acacia seyal (legumes group). After collection, the gum can be further broken down and be processed. Arabic gum is a natural source of fiber, mineral salts, and carbohydrates. It is water-soluble and contains potassium, magnesium, calcium, and carbohydrates called arabinose and galactose. Arabic gum is a complex combination of glycoproteins and polysaccharides and is primarily used in the food industry as a stabilizer. Arabic gum is widely used as a source of soluble fiber in diet drinks and is also used as a thickener and emulsifier in the beverage industry. Arabic gum used to be widely used in the pharmaceutical industry to fortify tablets and to concentrate some syrups.
2. Tragacanth Gum: Tragacanth gum, like Arabic gum, has an ancient history that is a dry exudate from the stems and branches of Astragalus gummifer (and other Asian species of Astragalus (legumes group)). The secretion of tragacanth gum is caused by injuring (pruning) various plants that are collected after drying. Tragacanth, when dissolved in water, forms a thick, sticky solution. This product is one of the most resistant gums to acid, especially in high viscosities in the range of 2 pH to 10. This makes it widely used in the pharmaceutical, food, and agricultural industries. It is also an excellent emulsion agent for oil-in-water emulsions.
3. Gellan Gum: One of the extracellular polysaccharides secreted by Pseudomonas elodea is gellan gum. Gellan is produced commercially by a fermentation process and is a linear, anionic exopolysaccharide, with the repeating unit containing D-glucose, D-glucuronate, and a-L-rhamnose. The form of gellan gum has two types of acyl substituents, namely, acetyl and L-glyceryl. It is a good substitution for other gelling agents because it is efficient in minimal amounts and creates a clear gel that is not susceptible to heat. It is used to stabilize, bind, or texturize processed foods in the food industry. Gellan gum has applications in various foods, including confectioneries, fruit and vegetable products, sauces and spreads, beverages, and packaged foods. Specific gelling attributes in various sciences led to the development of controlled-release formulation based on gellan. Different formulations have been studied, including ophthalmic, oral, and nasal. Recent reports showed that gellan-based materials could also be used in medicine, food, and agriculture sciences.
4. Xanthan Gum: Xanthan gum is produced using various compounds and inexpensive nutrients such as whey, sugarcane molasses, and sucrose. Xanthan gum is the first new generation of extracellular polymorphism in biotechnology produced by bacteria like Xanthomonas campestris. The constituent units of this gum are glucose, mannose, and di-glucuronic acid. Despite having high molecular weight, this gum dissolves easily in hot and cold water and, even in small amounts, produces a very concentrated solution. As a result of stirring, its viscosity decreases. Changes in the pH of the pewter have little effect on it. The gum is used in many industries, including chemicals, petroleum products, and cosmetics. This gum is used in various beverages, canned food, and frozen foods. Factors such as pH, temperature, high pressure, carbon sources, the effect of polymer concentration, and the development of salts and viscosity in the presence of galactomannan are influential in the production of xanthan gum. Various compounds and inexpensive nutrients are used to produce xanthan gum, such as whey, sugarcane molasses, and sucrose as a source of carbohydrates and ammonium and nitrate, yeast extract, and soy as a source of nitrogen.
5. Carrageenan Gum: Carrageenan gum is a polysaccharide with low molecular weight (16-44 kDa) containing ions of various metals, including K+, Na+, Ca2+, and Mg2+, and polysaccharide-protein in gum gives it emulsifying properties. Carrageenan gum from (Anacardium occidentale L.) is secreted. This gum is dissolved at room temperature; however, heating it improves its dissolution. Carrageenan gum can be an excellent alternative to Arabic gum.

### Other Polysaccharides Used Include Celluloses and Starch Derivatives:

Celluloses may be selected from but not limited to celluloses such as alkyl cellulose (methyl cellulose), a hydroxyalkyl cellulose (e.g., hydroxymethyl cellulose, Hydroxypropyl cellulose), carboxyalkyl cellulose (e.g., carboxymethyl cellulose and alkali metal salts thereof, such as sodium salts), a carboxyalkyl alkyl cellulose (e.g., carboxymethyl ethyl cellulose), carboxyalkyl cellulose ester or the combination thereof.

Polysaccharides may be selected from starch derivatives such as Modified Starch, corn starch, potato starch, pregelatinized starch, dextrin's, acid-treated starch, alkaline-treated starch, bleached starch, oxidized starch, enzyme-treated, monostarch phosphate, distarch phosphate, phosphate distarch phosphate, acetylated distarch phosphate, starch acetate, acetylated distarch adipate, hydroxypropyl starch, hydroxypropyl distarch phosphate, hydroxypropyl distarch glycerol, starch sodium octenyl succinate, acetylated oxidized starch and maltodextrin or the combination thereof.

Encapsulation is a promising approach that is widely used to overcome oxidation deterioration, unpleasant taste, stability, sensitivity to light and heat, which limits application of oil-based extracts in the food industry. Encapsulation may enhance ease of handling, adequacy of concentration and uniformity of dispersion as well. Encapsulation technologies are even used to improve the water solubility of regular herbal extracts.

Spray dryer drying, spray cooling, fluidized bed coating, extrusion method, centrifugal extrusion, coagulation, complexation, liposome entrapment, and rotational suspension separation are some of Encapsulation techniques. Emulsion, extrusion, and spray dryer are more critical among these methods. In the present invention pomegranate seed oil has been encapsulated together with other pomegranate extracts standardised to ellagic acid and punicalagins to yield a uniform water dispersible powder.

In an embodiment, the process steps are as follows:
Infusing all three bio actives of Pomegranate into a single ingredient format.
Combining active in oil format with powder extracts using the process technology.
Plant gum-based encapsulation to enhance the invitro stability, solubility and minimize the hydrolysis metabolism of Ellagic acid.
Enhancing the solubility/bioavailability and clinical efficacy of the formulation.

In an embodiment, encapsulation may be made from gum arabica, while in another embodiment encapsulation methodologies like Liposomal formats may be used to emulate this technology. Other technologies like nano emulsion or phytosome or any other techniques may be used to improve bioavailability of the above actives.

Advantages of the embodiments of the present invention include, a complete natural ingredient formula targeting high therapeutic benefits of plantextracts, a clean label pure aqueous based manufacturing process for targeted health wellness segments

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope.

The following examples given below are illustrative examples of the composition prepared in the form of a free-flowing powder with varying concentrations and ratios of the active ingredients

### EXAMPLES & MANUFACTURING PROCESS

### Example 1

| Composition | % w/w |
|---|---|
| Pomegranate extract (Ellagic acid) | 18.75 |
| Pomegranate extract (Punicalagins) | 25 |
| Pomegranate oil | 10 |
| Gum Arabica | 46.250 |
| P. water | q.s |

### Manufacturing Process—

Gum Arabica powder was dissolved in pre warm purified water under stirring Pomegranate extract powder were added into the above solution. Punicic acid oil base was added slowly to above solution and continue stirring to ensure a clear stable fine suspension is formed. The above material is being passed through colloidal mill and ensure a uniform homogenous suspension proceeded for spray drying to fine powder.

### Example 2

| Composition | % w/w |
|---|---|
| Pomegranate extract (Ellagic acid & | 43.75 |
| Punicalagins) | |
| Gum arabica | 44.25 |
| Pomegranate oil | 10 |
| Quillaia Extract | 2 |
| P. water | q.s |

Gum Arabica powder was dissolved in pre warm purified water under stirring Pomegranate extract powder were added into the above solution. Quillaia Extract was added to above solution followed by Punicic acid oil base was added slowly and continue stirring to ensure a clear stable fine suspension is formed. The above material is being passed through colloidal mill and ensure a uniform homogenous suspension proceeded for spray drying to fine powder.

### Example 3

| Composition | % w/w |
|---|---|
| Pomegranate extract (Ellagic acid & | 69 |
| Punicalagins) | |
| Gum arabica | 17 |
| Pomegranate oil | 13 |
| Sun flower lecithin | 1 |
| P. water | Q, s |

Gum Arabica powder was dissolved in pre warm purified water under stirring Pomegranate extract powder were added into the above solution. Sunflower Lecithin was added to above solution followed by Punicic acid oil base was added slowly and continue stirring to ensure a clear stable fine suspension is formed. The above material is being passed through colloidal mill and ensure a uniform homogenous suspension proceeded for spray drying to fine powder.

### Example 4

| Composition | % w/w |
|---|---|
| Pomegranate extract (Ellagic acid & | 43.75 |
| Punicalagins) | |
| Gum arabica | 30 |
| Pomegranate oil | 6.5 |
| Modified Starch | 18.25 |
| Polysorbate 80 | 1 |
| P. water | q.s |

Modified starch powder and Gum Arabica was dissolved in pre warm purified water under stirring. Pomegranate extract powder was added into the above solution. Polysorbate 80 was added to above solution followed by Punicic acid oil base was added slowly and continue stirring to ensure a clear stable fine suspension is formed. The above material is being passed through colloidal mill and ensure a uniform homogenous suspension proceeded for spray drying to fine powder.

### Example 5

| Composition | % w/w |
|---|---|
| Pomegranate extract (Ellagic acid & | 43.75 |
| Punicalagins) | |
| Gum arabica | 48.25 |
| Pomegranate oil | 6.5 |
| DL Alpha tocopherol | 0.5 |
| Polysorbate 80 | 1 |
| P. water | q.s |

Gum Arabica and DL alpha tocopherol were dissolved in pre warm purified water under stirring. Pomegranate extract powder was added into the above solution. Polysorbate 80 was added to above solution followed by Punicic acid oil base was added slowly and continue stirring to ensure a clear stable fine suspension is formed. The above material is being passed through colloidal mill and ensure a uniform homogenous suspension proceeded for spray drying to fine powder.

### Example 6

| Composition | % w/w |
|---|---|
| Pomegranate extract (Ellagic acid & | 43.75 |
| Punicalagins) | |
| Modified Starch | 44.25 |
| Pomegranate oil | 10 |
| Quillaia Extract | 2 |
| P. water | q.s. |

Modified starch was dissolved in pre warm purified water under stirring Pomegranate extract powder were added into the above solution. Quillaia Extract was added to the above solution followed by Punicic acid oil base was added slowly and continue stirring to ensure a clear stable fine suspension is formed. The above material is being passed through colloidal mill and ensure a uniform homogenous suspension proceeded for spray drying to fine powder.

### Example 7

| Composition | % w/w |
|---|---|
| Pomegranate extract (Ellagic acid & | 43.75 |
| Punicalagins) | |
| Modified Starch | 24.10 |
| Hydroxypropyl Methyl Cellulose | 20.15 |
| Pomegranate oil | 10 |
| Quillaia Extract | 2 |
| P. water | q.s. |

Modified starch was dissolved in pre warm purified water under stirring and Hydroxypropyl methyl cellulose added under stirring to form uniform solution. Pomegranate extract powder was added into the above solution. Quillaia Extract was added to above solution followed by Punicic acid oil base was added slowly and continue stirring to ensure a clear stable fine suspension is formed. The above material is being passed through colloidal mill and ensure a uniform homogenous suspension proceeded for spray drying to fine powder.

A typical formulation used for a clinical study is depicted below:

| Composition | % w/w | Mg |
|---|---|---|
| Pomegranate extract | 18.75 | 56.25 |
| (Ellagic acid) | | |
| Pomegranate extract | 25 | 75.00 |
| (Punicalagins) | | |
| Pomegranate oil | 10 | 30.00 |
| Gum Arabica | 46.250 | 138.75 |
| P. water | q.s | 300 mg |

One of the above compositions were filled into hard gelatin capsules at a dose of 300 mg and submitted for Human clinical studies. A randomized placebo controlled clinical study was carried out on 80 Healthy subjects and evaluated the parameters by way of scoring like crow's feet wrinkle, skin tactile roughness, skin redness, forehead wrinkles, skin elasticity and firmness, skin hydration level, skin pores and change in UV pigmentation. Secondary Objectives of the study included assessment of the safety of test product through evaluation and reporting of adverse events during the study.

The clinical efficacy study carried out on healthy volunteers indicates skin health benefits of the combination extract in an optimized ratio at a dose of 300 mg per day supplementation for 60 days.

The results of this randomized, double-blind, placebo-controlled study demonstrated that the product studied improves overall skin appearance, skin glow, wrinkles and helps regulating hyper pigmentation. Further, present study underlines the positive effect of this unique pomegranate-based ingredient in improving facial pores, spots, evenness, and Skin elasticity levels at a dose of 300 mg. The study is unique with a biomarker (Tyrosinase) measurement to understand the impact of test product in regulating melanin synthesis pathways. The test composition inhibits the Tyrosinase enzymes and there by reduces hyper pigmentation.

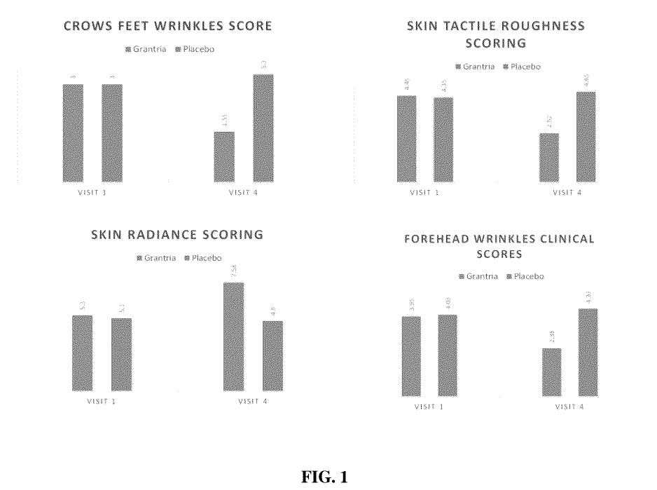

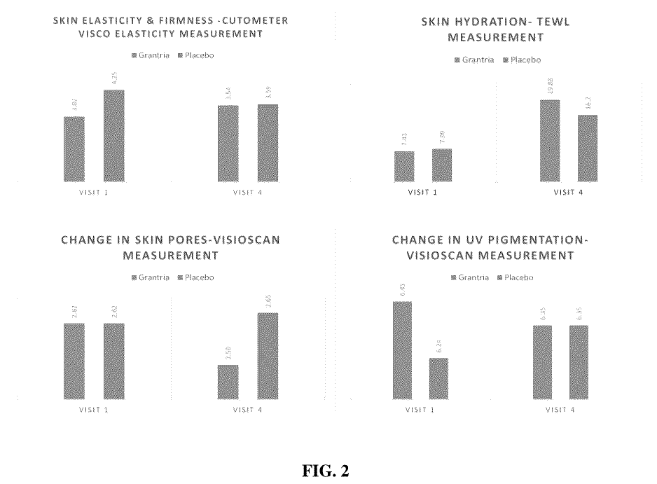

The results of the clinical study and differences in score on all the parameters listed above during Visit 1 and Visit 4 are depicted graphically in FIG. 1 and FIG. 2

The study was conducted on 35-65 years healthy female adults and the test product efficacy, safety compared against placebo during 60 day supplementation.

Crow's Feet, also known as smile lines, are very common wrinkle that form at the corners of the eyes. Crow's feet wrinkles tend to initially appear in the mid to late 30 s. The crow's feet wrinkles scores for test product dropped significantly from an average of 3 to 1.55 during the supplementation with Test product, where as the placebo group didn't show any improvement.

The tactile roughness score of the face under an artificial daylight system was measured by the dermatologist and observed to have a significant improvement of around 43.14% at visit 4 compared to Visit 1 (p<0.001) for test group. In this case also, Placebo group did not show any changes between visit 1 average score of 4.35 against visit 4 score of 4.65.

The decrease in the tactile roughness values in the subjects treated with the test product underlines the effectiveness in modulating skin smoothness with reduced wrinkles.

The Skin radiance score indicates the effectiveness of product in improving skin glow. The scores reflect a statistically significant difference was observed between test product and placebo (p<0.001) with test group has shown an improvement of 42.8% in 60 days. The changes from Visit 1 to visit 4 was also observed to be statistically significant (p<0.001). The placebo group didn't reflect any positive changes from visit 1 to 4.

The Scoring for forehead fine lines was done by the dermatologist from grade 0 to 7 for the female subjects using a skin aging atlas under an artificial daylight system. The average clinical score of forehead fine lines were decreased by 45% for test group from Visit 1 to visit 4, however the placebo group exhibited negative changes.

The skin elasticity and firmness measured by cutometer VE (Visco Elasticity, MPa) reflected an improvement of around 17.2% in test supplemented group, though the changes were not statistically significant.

The changes in skin moisturization levels were evaluated by corneometer and test group and placebo group exhibited similar changes from visit 1 to visit 4. The changes were not statistically significant.

The Changes in Skin Hydration levels were assessed using Tewameter (Transepidermal water loss (TEWL)) and both groups exhibited non-significant changes post supplementation. TEWL is the amount of water vapor evaporating from the surface of the skin. In healthy skin, TEWL is directly proportional to skin hydration.

The UV pigmentation score measured by Visio Scan indicates a slightly improvement of around 1.2% in subjects treated with the test product and in case of placebo the changes were on negative side.

The assessment readings for Skin pores, Skin tone evenness, Spots, wrinkles were measured by the instrument Visio scan. The Desired evaluation area selected by dermatologist was marked using repositioning sheets and one defined zone of face (left cheek/right cheek) was used on all visits. The study results indicate that the test group has shown statistically significant improvement in these parameters at the end of Visit 4 as compared to Visit 1.

Further the clinical efficacy of test product in altering melanin pathways were also measured by using the biomarker Tyrosinase. Melanin is derived from the amino acid—tyrosine—through a synthesis regulated by the enzyme tyrosinase. Thus, the regulation of melanin synthesis by inhibiting the tyrosinase enzyme plays a major role in preventing hyperpigmentation. The test product has shown a drop of around 3% in tyrosinase levels which could support in improving the skin glow.

There were no adverse events reported to the test product during the study. Thus, the test product was efficacious and safe, and well tolerated by the study subjects.

This ratio which is clinically effective and available in a powdered form by virtue of the process described herein above, comprising the three constituents namely ellagic acid, punicalagin and punicic acid can be formulated into a variety a of dosage forms like tablets, capsules, granules, effervescent tablets, oral solutions, suspensions, emulsion or also incorporated into topical products like creams, ointments, face masks, face peels and similar other applications.

We claim:

1. A solid powder composition comprising ellagic acid, punicalagin and punicic acid to achieve a substantially aqueous soluble formulation comprising ellagic acid from 10 to 40% w/w, punicalagin from 25 to 50% w/w and punicic acid from 5 to 20% w/w.

2. A solid powder composition comprising ellagic acid, punicalagin and punicic acid to achieve a substantially aqueous soluble product having an optimized ratio of 1:0.5:0.1 to 1:1.5:0.7 (ellagic acid:punicalagin:punicic acid).

3. A composition of claim 2, wherein the preferred ratio is 1:0.6:0.3 to 1:1.33:0.6.

4. A composition of claim 1, wherein in case combined ellagic acid and punicalagin is used then the composition comprises ellagic acid and puninicalagin (combined) from 35 to 90% w/w and punicic acid from 5 to 20% w/w.

5. A composition of claim 4, wherein in case combined ellagic acid and punicalagin is used then the combined ratio of the two ingredients to punicic acid is optimized in the range of 1:0.1 to 1:0.5.

6. A composition of claim 5, wherein the preferred ratio is from 1:0.15 to 1:0.3.

7. A solid oral composition of claim 1 comprising a natural gum or a polysaccharide is used as an encapsulating agent.

8. A composition of claim 7, wherein the natural gum is gum arabica.

9. A composition of claim 7, wherein the polysaccharide is modified starch and/or HPMC.

10. A composition of claim 1, optionally comprising additional excipients like vitamins, extracts, and pharmaceutical fillers, glidants.

11. A process for manufacture of a solid powder composition comprising the steps of:
a. Dissolving gum arabica/polysaccharide to pre-heated water to get a clear solution.
b. Dissolving the extract comprising ellagic acid and punicalagin or individual extracts of ellagic acid and punicalagin to step (a) solution under stirring till a clear solution is formed.
c. Add Pomegranate seed oil standardized to Punicic acid to the step (b) solution slowly.
d. Homogenization and colloidal milling at an optimal speed & time of the step (c) solution
e. Spray drying the solution of step (d) to obtain a water-soluble powder containing the combined extracts in the optimized ratio.
f. Sifting and uniform blending of the powder obtained in step (e).

12. A composition for oral administration comprising the powdered blend of claim 1, 2 or claim 3.

13. A composition for topical application comprising the powdered blend of claim 1, 2 or claim 3.

14. A composition of claim 1, 2 or claim 3 providing healthcare benefits.
